(19) United States
(12) Patent Application Publication
(10) Pub. No.: US 2017/0209425 A1
(43) Pub. Date: Jul. 27, 2017

(54) METHODS AND COMPOSITIONS FOR TREATING PATHOLOGIES ASSOCIATED WITH BDNF SIGNALING

(71) Applicant: CASE WESTERN RESERVE UNIVERSITY, Cleveland, OH (US)

(72) Inventor: David M. Katz, Cleveland, OH (US)

(21) Appl. No.: 15/284,388

(22) Filed: Oct. 3, 2016

Related U.S. Application Data
(63) Continuation of application No. 12/306,552, filed on Oct. 16, 2009, now abandoned.
(60) Provisional application No. 60/816,547, filed on Jun. 26, 2006.

(51) Int. Cl. A61K 31/453

(52) U.S. Cl. CPC A61K 31/453

(57) ABSTRACT

A method of treating non-neurodegenerative pathologies associated with derangement in brain-derived neurotrophic factor signaling in the brain stem includes administering to the subject an amount of at least one ampakine effective to increase brain-derived neurotrophic factor nodose sensory neurons of the subject.

### RELATED APPLICATION

This application claims priority from U.S. Provisional Application No. 60/816,547, filed Jun. 26, 2006, the subject matter, which is incorporated herein by reference.

### GOVERNMENT FUNDING

This invention was made with government support under Grant No. NIH-HL042131-16 awarded by National Institute of Health. The government has certain rights in the invention.

### FIELD OF THE INVENTION

The present invention relates to methods and compositions used for treating pathologies associated with brain-derived neurotrophic signaling and particularly relates to the use of ampakines for the treatment of Rett syndrome.

### BACKGROUND

Rett Syndrome (RTT) is a neurodevelopmental disorder that is classified as a pervasive developmental disorder. Pervasive development disorders refers to a group of disorders characterized by delays in the development of multiple basic functions including socialization and communication. RTT is caused by loss-of-function mutations in the gene encoding the methyl-CpG binding protein MeCP2 and is characterized by severe mental retardation and somatomotor and autonomic dysfunction. Abnormal expression of Brain-Derived Neurotrophic Factor (BDNF) has been highlighted as a possible cause of neurologic dysfunction in RTT. However, no studies have examined how genetic loss of MeCP2 affects transynaptic BDNF signaling, a highly regulated process that requires tight coupling between activity dependent BDNF expression and secretion presynaptically as well as expression and activation of BDNF receptors postsynaptically.

### SUMMARY OF THE INVENTION

The present invention relates to a method of treating non-neurodegenerative pathologies associated with derangement in brain-derived neurotrophic factor signaling in the brain stem. In the method, an amount of at least one ampakine effective to increase brain-derived neurotrophic factor expression in nodose sensory neurons of the subject is administered to the subject. The non-neurodegenerative pathology can be a pervasive developmental disorder. In one example, the non-neurodegenerative pathology can include respiratory abnormalities associated with the pervasive developmental disorder and the amount of ampakine administered to subject can be that amount effective to improve respiratory function of the subject.

In an aspect of the invention the ampakine can be an allosteric modulator of the AMPA-receptor. The allosteric modulator of the AMPA-receptor can comprise a compound having the formula:

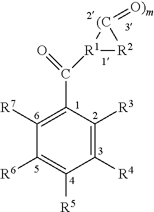

wherein,

R¹ is a member selected from the group consisting of N and CH;

m is 0 or 1;

R² is a member selected from the group consisting of (CR⁸₂)ₙ₋ₘ and Cₙ₋ₘR⁸₂₍ₙ₋ₘ₎₋₂, in which n is 4, 5, 6, or 7, the R⁸'s in any single compound being the same or different, each R⁸ being a member selected from the group consisting of H and C₁-C₆ alkyl, or one R⁸ being combined with either R³ or R⁷ to form a single bond linking the no. 3′ ring vertex to either the no. 2 or the no. 6 ring vertices or a single divalent linking moiety linking the no. 3′ ring vertex to either the no. 2 or the no. 6 ring vertices, the linking moiety being a member selected from the group consisting of CH₂, CH₂CH₂, CH═CH, O, NH, N(C₁-C₆ alkyl), N═CH, N═C(C₁-C₆ alkyl), C(O), O—C(O), C(O)—O, CH(OH), NH—C(O), and N(C₁-C₆ alkyl)-C(O);

R³, when not combined with any R⁸, is a member selected from the group consisting of H, C₁-C₆ alkyl, and C₁-C₆ alkoxy;

R⁴ is either combined with R⁵ or is a member selected from the group consisting of H, OH, and C₁-C₆ alkoxy;

R⁵ is either combined with R⁴ or is a member selected from the group consisting of H, OH, C₁-C₆ alkoxy, amino, mono(C₁-C₆ alkyl)amino, di(C₁-C₆ alkyl)amino, and CH₂OR⁹, in which R⁹ is a member selected from the group consisting of H, C₁-C₆ alkyl, an aromatic carbocyclic moiety, an aromatic heterocyclic moiety, an aromatic carbocyclic alkyl moiety, an aromatic heterocyclic alkyl moiety, and any such moiety substituted with one or more members selected from the group consisting of C₁-C₃ alkyl, C₁-C₃ alkoxy, hydroxy, halo, amino, alkylamino, dialkylamino, and methylenedioxy;

R⁶ is either H or CH₂OR⁹;

R⁴ and R⁵ when combined form a member selected from the group consisting of

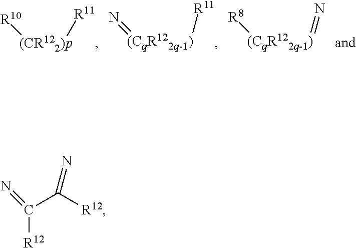

in which: R¹⁰ is a member selected from the group consisting of O, NH and N(C₁-C₆ alkyl);

R¹¹ is a member selected from the group consisting of O, NH and N(C₁-C₆ alkyl);

R¹² is a member selected from the group consisting of H and C₁-C₆ alkyl, and when two or more R¹²'s are present in a single compound, such R¹²'s are the same or different;

p is 1, 2, or 3; and

q is 1 or 2; and

R⁷, when not combined with any R⁸, is a member selected from the group consisting of H, C₁-C₆ alkyl, and C₁-C₆ alkoxy.

In a further aspect, the ampakine can comprise at least one of 1-(1,4-benzodioxan-6-ylcarbonyl)piperidine, 1-(quinoxalin-6-ylcarbonyl)piperidine, and 2H,3H,6aH-pyrrolidino[2″,1″-3′,2″]1,3-oxazino[6′,5′-5,4]benzo[e]1,4-dioxan-10-one.

The amount of the ampakine administered to subject can be, for example, about 10 mg/kg per day to about 50 mg/kg per day. The ampakine can be delivered in a single dose or multiple doses administered periodically throughout the day.

The present invention also relates to a method of treating non-neurodegenerative respiratory disorders in a subject caused by Rett syndrome. The method can include administering to the subject an amount of at least one ampakine effective to increase brain-derived neurotrophic factor expression in nodose sensory neurons

In an aspect of the invention the ampakine can be an allosteric modulator of the AMPA-receptor. The allosteric modulator of the AMPA-receptor can comprise a compound having the formula:

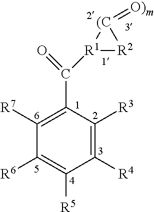

wherein,

R¹ is a member selected from the group consisting of N and CH;

m is 0 or 1;

R² is a member selected from the group consisting of (CR⁸₂)ₙ₋ₘ and Cₙ₋ₘR⁸₂₍ₙ₋ₘ₎₋₂, in which n is 4, 5, 6, or 7, the R⁸'s in any single compound being the same or different, each R⁸ being a member selected from the group consisting of H and C₁-C₆ alkyl, or one R⁸ being combined with either R³ or R⁷ to form a single bond linking the no. 3′ ring vertex to either the no. 2 or the no. 6 ring vertices or a single divalent linking moiety linking the no. 3′ ring vertex to either the no. 2 or the no. 6 ring vertices, the linking moiety being a member selected from the group consisting of CH₂, CH₂CH₂, CH═CH, O, NH, N(C₁-C₆ alkyl), N═CH, N═C(C₁-C₆ alkyl), C(O), O—C(O), C(O)—O, CH(OH), NH—C(O), and N(C₁-C₆ alkyl)-C(O);

R³, when not combined with any R⁸, is a member selected from the group consisting of H, C₁-C₆ alkyl, and C₁-C₆ alkoxy;

R⁴ is either combined with R⁵ or is a member selected from the group consisting of H, OH, and C₁-C₆ alkoxy;

R⁵ is either combined with R⁴ or is a member selected from the group consisting of H, OH, C₁-C₆ alkoxy, amino, mono(C₁-C₆ alkyl)amino, di(C₁-C₆ alkyl)amino, and CH₂OR⁹, in which R⁹ is a member selected from the group consisting of H, C₁-C₆ alkyl, an aromatic carbocyclic moiety, an aromatic heterocyclic moiety, an aromatic carbocyclic alkyl moiety, an aromatic heterocyclic alkyl moiety, and any such moiety substituted with one or more members selected from the group consisting of C₁-C₃ alkyl, C₁-C₃ alkoxy, hydroxy, halo, amino, alkylamino, dialkylamino, and methylenedioxy;

R⁶ is either H or CH₂OR⁹;

R⁴ and R⁵ when combined form a member selected from the group consisting of

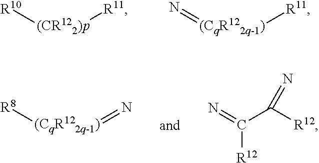

in which: R¹⁰ is a member selected from the group consisting of O, NH and N(C₁-C₆ alkyl);

R¹¹ is a member selected from the group consisting of O, NH and N(C₁-C₆ alkyl);

R¹² is a member selected from the group consisting of H and C₁-C₆ alkyl, and when two or more R¹²'s are present in a single compound, such R¹²'s are the same or different;

p is 1, 2, or 3; and

q is 1 or 2; and

R⁷, when not combined with any R⁸, is a member selected from the group consisting of H, C₁-C₆ alkyl, and C₁-C₆ alkoxy.

In a further aspect, the ampakine can comprise at least one of 1-(1,4-benzodioxan-6-ylcarbonyl)piperidine, 1-(quinoxalin-6-ylcarbonyl)piperidine, and 2H,3H,6aH-pyrrolidino[2″,1″-3′,2″]1,3-oxazino[6′,5′-5,4]benzo[e]1,4-dioxan-10-one.

The amount of the ampakine administered to subject can be, for example, about 10 mg/kg per day to about 50 mg/kg per day. The ampakine can be delivered in a single dose or multiple doses given periodically throughout the day.

The present invention also relates to a method of treating respiratory disorders in a subject caused by loss-of-function mutations of the gene encoding methyl-CpG binding protein 2 (MeCP2). The method can include administering to the subject an amount of at least one ampakine effective to increase brain-derived neurotrophic factor expression in nodose sensory neurons. In an aspect of the invention, the respiratory disorder may be a non-neurodegenerative pathology of Rett Syndrome.

In an aspect of the invention the ampakine can be an allosteric modulator of the AMPA-receptor. The allosteric modulator of the AMPA-receptor can comprise a compound having the formula:

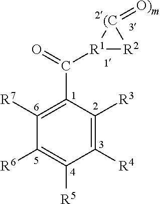

wherein,

R¹ is a member selected from the group consisting of N and CH;

m is 0 or 1;

R² is a member selected from the group consisting of (CR⁸₂)ₙ₋ₘ and Cₙ₋ₘR⁸₂₍ₙ₋ₘ₎₋₂, in which n is 4, 5, 6, or 7, the R⁸'s in any single compound being the same or different, each R⁸ being a member selected from the group consisting of H and C₁-C₆ alkyl, or one R⁸ being combined with either R³ or R⁷ to form a single bond linking the no. 3′ ring vertex to either the no. 2 or the no. 6 ring vertices or a single divalent linking moiety linking the no. 3′ ring vertex to either the no. 2 or the no. 6 ring vertices, the linking moiety being a member selected from the group consisting of CH₂, CH₂CH₂, CH═CH, O, NH, N(C₁-C₆ alkyl), N═CH, N═C(C₁-C₆ alkyl), C(O), O—C(O), C(O)—O, CH(OH), NH—C(O), and N(C₁-C₆ alkyl)-C(O);

R³, when not combined with any R⁸, is a member selected from the group consisting of H, C₁-C₆ alkyl, and C₁-C₆ alkoxy;

R⁴ is either combined with R⁵ or is a member selected from the group consisting of H, OH, and C₁-C₆ alkoxy;

R⁵ is either combined with R⁴ or is a member selected from the group consisting of H, OH, C₁-C₆ alkoxy, amino, mono(C₁-C₆ alkyl)amino, di(C₁-C₆ alkyl)amino, and CH₂OR⁹, in which R⁹ is a member selected from the group consisting of H, C₁-C₆ alkyl, an aromatic carbocyclic moiety, an aromatic heterocyclic moiety, an aromatic carbocyclic alkyl moiety, an aromatic heterocyclic alkyl moiety, and any such moiety substituted with one or more members selected from the group consisting of C₁-C₃ alkyl, C₁-C₃ alkoxy, hydroxy, halo, amino, alkylamino, dialkylamino, and methylenedioxy;

R⁶ is either H or CH₂OR⁹;

R⁴ and R⁵ when combined form a member selected from the group consisting of

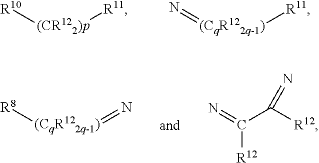

in which: R¹⁰ is a member selected from the group consisting of O, NH and N(C₁-C₆ alkyl);

R¹¹ is a member selected from the group consisting of O, NH and N(C₁-C₆ alkyl);

R¹² is a member selected from the group consisting of H and C₁-C₆ alkyl, and when two or more R¹²'s are present in a single compound, such R¹²'s are the same or different;

p is 1, 2, or 3; and

q is 1 or 2; and

R⁷, when not combined with any R⁷, is a member selected from the group consisting of H, C₁-C₆ alkyl, and C₁-C₆ alkoxy.

In a further aspect, the ampakine can comprise at least one of 1-(1,4-benzodioxan-6-ylcarbonyl)piperidine, 1-(quinoxalin-6-ylcarbonyl)piperidine, and 2H,3H,6aH-pyrrolidino[2″,1″-3′,2″]1,3-oxazino[6′,5′-5,4]benzo[e]1,4-dioxan-10-one.

The amount of the ampakine administered to subject can be, for example, about 10 mg/kg per day to about 50 mg/kg per day. The ampakine can be delivered in a single dose or multiple doses given periodically throughout the day.

### BRIEF DESCRIPTION OF THE DRAWINGS

FIG. 1 is a photograph illustrating that MeCP2 protein is expressed in nodose neurons. Double-immunostaining for MeCP2 and β3-tubulin in the newborn wildtype (Mecp2^{+/y}) mouse nodose ganglion (NG). The right panel is a higher magnification of the same section shown on the left and illustrates the concentration of MeCP2 immunoreactive protein in heterochromatin foci. The insert in the right panel shows that the MeCP2 antibody used in these studies does not produce any specific staining in the NG from a Mecp2 null mouse (Mecp2^{+/y}).

FIG. 2 are charts illustrating BDNF levels are depressed in P35 Mecp2^{+/y} NG neurons and can be increased by KCl depolarization. Summary data showing that BDNF content is decreased by 40-50% in NG cultures from Mecp2 null mutants, regardless of the activity state of the cells, i.e., electrically silent (A, treated with TTX) or chronic depolarization (C, treated with KCl). Results show that KCl treatment can increase BDNF level in mutant cells as in wildtype controls. Neuron survival was unaffected by either TT′X (B) or KCl (D). Results are the mean±SEM (n=6). **p<0.01, ANOVA I with post-hoc Tukey test.

FIG. 3 are plethysmographic recordings from wildtype (Mecp2^{+/y}) and Mecp2 null mice (Mecp2^{+/y}) that show Mecp2 null mice exhibit a Rett-like respiratory phenotype at 5 weeks of age (P35). Each trace=10 s quiet breathing in room air. Lower graphs are frequency histograms from control (compilation of 9776 breath cycles) and mutant (compilation of 6065 breath cycles) mice showing the higher incidence of fast breaths in mutant mice compared to controls, along with a shift to higher values of minute volume/weight.

FIG. 4 illustrates histograms that show chronic treatment with CX546 restores normal breathing frequency and minute volume/weight in P35 Mecp2 null mice. Representative histograms of breathing frequency (A) and minute volume/weight (B) from two mutant mice; one treated with vehicle (9227 breath cycles), and one treated with CX546 (8393 breath cycles), showing that drug treatment (40 mg/kg, b.i.d for 3 days) decreases episodes of high breathing frequency and minute volume/weight. Summary data for breathing frequency and minute volume/weight for all animals are shown in (C) and (D), respectively. Ampakine treatment completely restores wildtype frequency and minute volume/weight in mutant animals and has no effect in wildtypes. Results are the mean±SEM (n=8 for vehicle-treated wildtypes, n7 for CX546-treated wildtypes, n=8 for vehicle-treated mutants and n=9 for CX546-treated mutants). *p<0.05, **p<0.01, ANOVA I with post-hoc Tukey test.

### DETAILED DESCRIPTION

As used herein, the tern “therapeutically effective amount” refers to that amount of a composition that results in amelioration of symptoms or a prolongation of survival in a patient. A therapeutically relevant effect relieves to some extent one or more symptoms of a disease or condition or returns to normal either partially or completely one or more physiological or biochemical parameters associated with or causative of the disease or condition.

As used herein, the terms “host” and “subject” refer to any animal, including, but not limited to, humans and non-human animals (e.g., rodents, arthropods, insects, fish (e.g., zebrafish), non-human primates, ovines, bovines, ruminants, lagomorphs, porcines, caprines, equines, canines, felines, aves, etc.), which is to be the recipient of a particular treatment. Typically, the terms “host,” “patient,” and “subject” are used interchangeably herein in reference to a human subject.

As used herein, the terms “subject suffering from Rett syndrome”, “subject having Rett syndrome” or “subjects identified with Rett syndrome” refers to subjects that are identified as having or likely having a loss-of-function mutation in the gene encoding the methyl-CpG binding protein MeCP2 gene, which causes Rett syndrome.

The term “biologically active,” as used herein, refers to a protein or other biologically active molecules (e.g., catalytic RNA) having structural, regulatory, or biochemical functions of a naturally occurring molecule.

The term “modulate,” as used herein, refers to a change in the biological activity of a biologically active molecule. Modulation can be an increase or a decrease in activity, a change in binding characteristics, or any other change in the; biological, functional, or immunological properties of biologically active molecules.

As used herein, the term “in vitro” refers to an artificial environment and to processes or reactions that occur within an artificial environment. In vitro environments consist of, but are not limited to, test tubes and cell culture. The term “in vivo” refers to the natural environment (e.g., an animal or a cell) and to processes or reaction that occur within a natural environment.

The term “test compound” refers to any chemical entity, pharmaceutical, drug, and the like that are used to treat or prevent a disease, illness) sickness or disorder of bodily function. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms. Treatment, prevention and ameliorating a condition, as used herein, can include, for example decreasing or eradicating a deleterious or harmful condition associated with Rett syndrome. Examples of such treatment include: decreasing breathing abnormalities, decreasing motor dysfunction, and improving respiratory and neurological function.

“Cyano” refers to the group —CN.

“Halogen” or “halo” refers to fluorine, bromine, chlorine, and iodine atoms.

“Hydroxy” refers to the group —OH.

“Thiol” or “mercapto” refers to the group —SH.

“Sulfamoyl” refers to the —SO₂NH₂.

“Alkyl” refers to a cyclic, branched or straight chain, alkyl group of one to eight carbon atoms. The term “alkyl” includes reference to both substituted and unsubstituted alkyl groups. This term is further exemplified by such groups as methyl, ethyl, n-propyl, i-propyl, n-butyl, t-butyl, i-butyl (or 2-methylpropyl), cyclopropylmethyl, cyclohexyl, i-amyl, n-amyl, and hexyl. Substituted alkyl refers to alkyl as just described including one or more functional groups such as aryl, acyl, halogen, hydroxyl, amido, amino, acylamino, acyloxy, alkoxy, cyano, nitro, thioalkyl, mercapto and the like. These groups may be attached to any carbon atom of the lower alkyl moiety. “Lower alkyl” refers to C₁-C₆ alkyl, with C₁-C₄ alkyl more preferred. “Cyclic alkyl” includes both mono-cyclic alkyls, such as cyclohexyl, and bi-cyclic alkyls, such as bicyclooctane and bicycloheptane. “Fluoroalkyl” refers to alkyl as just described, wherein some or all of the hydrogens have been replaced with fluorine (e.g., —CF₃ or —CF₂CF₃).

“Aryl” or “Ar” refers to an aromatic substituent which may be a single ring or multiple rings which are fused together, linked covalently, or linked to a common group such as an ethylene or methylene moiety. The aromatic ring(s) may contain a heteroatom, such as phenyl, naphthyl, biphenyl, diphenylmethyl, 2,2-diphenyl-1-ethyl, thienyl, pyridyl and quinoxalyl. The term “aryl” or “Ar” includes reference to both substituted and unsubstituted aryl groups. If substituted, the aryl group may be substituted with halogen atoms, or other groups such as hydroxy, cyano, nitro, carboxyl, alkoxy, phenoxy, fluoroalkyl and the like. Additionally, the aryl group may be attached to other moieties at any position on the aryl radical which would otherwise be occupied by a hydrogen atom (such as 2-pyridyl, 3-pyridyl and 4-pyridyl).

The term “alkoxy” denotes the group —OR, where R is lower alkyl, substituted lower alkyl, aryl, substituted aryl, aralkyl or substituted aralkyl as defined below.

The term “acyl” denotes groups —C(O)R, where R is alkyl, substituted alkyl, alkoxy, aryl, substituted aryl, amino and alkylthiol.

“Carbocyclic moiety” denotes a ring structure in which all ring vertices are carbon atoms. The term encompasses both single ring structures and fused ring structures. Examples of aromatic carbocyclic moieties are phenyl and naphthyl.

“Heterocyclic moiety” denotes a ring structure in which one or more ring vertices are atoms other than carbon atoms, the remainder being carbon atoms. Examples of non-carbon atoms are N, O, and S. The term encompasses both single ring structures and fused ring structures. Examples of aromatic heterocyclic moieties are pyridyl, pyrazinyl, pyrimidinyl, quinazolyl, isoquinazolyl, benzofuryl, isobenzofuryl, benzothiofuryl, indolyl, and indolizinyl.

The term “amino” denotes the group NRR′, where R and R′ may independently be hydrogen, lower alkyl, substituted lower alkyl, aryl, substituted aryl as defined below or acyl.

The term “amido” denotes the group —C(O)NRR′, where R and R′ may independently be hydrogen, lower alkyl, substituted lower alkyl, aryl, substituted aryl as defined below or acyl.

The term “independently selected” is used herein to indicate that the two R groups, R¹ and R², may be identical or different (e.g., both R¹ and R² may be halogen or, R¹ may be halogen and R² may be hydrogen, etc.).

“α-amino-3-hydroxy-5-methyl-isoxazole-4-propionic acid”, or “AMPA”, or “glutamatergic” receptors are molecules or complexes of molecules present in cells, particularly neurons, usually at their surface membrane, that recognize and bind to glutamate or AMPA. The binding of AMPA or glutamate to an AMPA receptor normally gives rise to a series of molecular events or reactions that result in a biological response. The biological response may be the activation or potentiation of a nervous impulse, changes in cellular secretion or metabolism, or causing cells to undergo differentiation or movement.

The present invention relates to a method of treating non-neurodegenerative pathologies associated with derangement in brain-derived neurotrophic factor signaling in the brain stem. By non-neurodegenerative pathology, it is means diseases and disorders that are not associated with neuronal injury or neuronal death. Pathologies that are non-neurodegenerative can include pathologies that are associated with impaired neurodevelopment. These non-neurodegenerative pathologies can include pervasive development disorders, such as Rett Syndrome and autism.

It was discovered that autonomic dysfunction, such as cardiorespiratory disturbances, including respiratory dysrhythmia, cardiac vagal tone, and cardiac baroreflex, is associated with derangement in BDNF signaling in the brainstem nucleus tractus solitarius (nTS). For example, in subjects with Rett Syndrome (RTT), genetic loss of the MeCP2 gene disrupts BDNF signaling in the nTS. It was found that normally visceral sensory neurons, located in the nodose cranial sensory ganglia (NG), synthesize and release high levels of BDNF, but that in MeCP2 null mice exhibiting respiratory dysfunction, the level of BDNF in the brain stem is substantially reduced. Surprisingly, it was also found that the level of BDNF in the cortex and hippocampus was not substantially impaired in MeCP2 null mice. The expression of BDNF from NG neurons was found to be increased by the administration of at least one ampakine to the NG neurons of MeCP2 null mice. The increase in expression of BDNF from the NG neurons is believed to improve synaptic transmission in MeCP2 null mice and improve or enhance respiratory function, characterized by at least partial restoration of normal breathing.

One aspect of the invention, therefore, relates to therapeutic agents that enhance BDNF expression in a subset of neurons that are important for respiratory and autonomic control (nodose cranial sensory ganglion cells (or NG neurons)) and, in addition, restore normal respiratory frequency and respiratory minute volume (frequency×tidal volume) caused by non-neurodegenerative pathologies in which BDNF synaptic transmission is impaired or derganged in the brain stem of the subject.

The invention is particularly based on the discovery that an effective amount of compounds of the ampakine family can be administered to a subject to potentially enhance BDNF expression levels from NG neurons in the subject and improve respiratory and neurological function in the subject. Applications contemplated for ampakines include improving the performance of subjects with sensory-motor problems, autonomic problems, gastrointestinal problems, and cardorespiratory problems dependent upon or associated with impaired or deranged BDNF synaptic transmission in the brain stem; improving the performance of subjects impaired in cognitive tasks dependent upon impaired or deranged BDNF synaptic transmission in the brain stem; improving the performance of subjects with memory deficiencies; and the like associated with impaired or deranged BDNF synaptic transmission in the brain stem. Additional applications contemplated for ampakines include restoring biochemical and synaptic transmission due to non-neurodegenerative pathologies, which are associated with a decrease in BDNF levels in the brain stem. Such therapeutic uses would include, but are not limited to, treating subjects with Rett syndrome by enhancing BDNF levels in NG neurons of the brain stem.

The at least one ampakine administered to the subject can be an allosteric modulator of the AMPA-receptor. Allosteric modulators of the AMPA-receptor that can be used for practicing the present invention and methods of making these compounds are disclosed in U.S. Pat. Nos. 5,488,049; 5,650,409; 5,736,543; 5,747,492; 5,773,434; 5,891,876; 6,030,968; 6,274,600 B1; 6,329,368 B1; 6,943,159 B1; 7,026,475 B2 and U.S. Application 20020055508. The disclosures of these publications are incorporated herein by reference in their entireties, especially with respect to the ampakines disclosed therein, which may be

The ampakine compound can include those compounds having the following general Formula I:

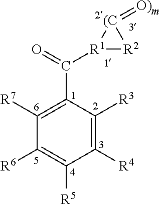

In this formula:

R¹ is a member selected from the group consisting of N and CH;

m is 0 or 1;

R² is a member selected from the group consisting of (CR⁸₂)ₙ₋ₘ and Cₙ₋ₘR⁸₂₍ₙ₋ₘ₎₋₂, in which n is 4, 5, 6, or 7, the R⁸'s in any single compound being the same or different, each R⁸ being a member selected from the group consisting of H and C₁-C₆ alkyl, or one R⁸ being combined with either R³ or R⁷ to form a single bond linking the no. 3′ ring vertex to either the no. 2 or the no. 6 ring vertices or a single divalent linking moiety linking the no. 3′ ring vertex to either the no. 2 or the no. 6 ring vertices, the linking moiety being a member selected from the group consisting of CH₂, CH₂CH₂, CH═CH, O, NH, N(C₁-C₆ alkyl), N═CH, N═C(C₁-C₆ alkyl), C(O), O—C(O), C(O)—O, CH(OH), NH—C(O), and N(C₁-C₆ alkyl)-C(O);

R³, when not combined with any R⁸, is a member selected from the group consisting of H, C₁-C₆ alkyl, and C₁-C₆ alkoxy;

R⁴ is either combined with R⁵ or is a member selected from the group consisting of H, OH, and C₁-C₆ alkoxy;

R⁵ is either combined with R⁴ or is a member selected from the group consisting of H, OH, C₁-C₆ alkoxy, amino, mono(C₁-C₆ alkyl)amino, di(C₁-C₆ alkyl)amino, and CH₂OR⁹, in which R⁹ is a member selected from the group consisting of H, C₁-C₆ alkyl, an aromatic carbocyclic moiety, an aromatic heterocyclic moiety, an aromatic carbocyclic alkyl moiety, an aromatic heterocyclic alkyl moiety, and any such moiety substituted with one or more members selected from the group consisting of C₁-C₃ alkyl, C₁-C₃ alkoxy, hydroxy, halo, amino, alkylamino, dialkylamino, and methylenedioxy; R⁶ is either H or CH₂OR⁹;

R⁴ and R⁵ when combined form a member selected from the group consisting of

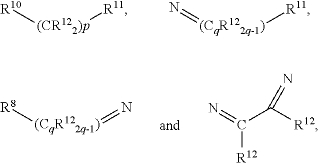

in which: R¹⁰ is a member selected from the group consisting of O, NH and N(C₁-C₆ alkyl);

R¹¹ is a member selected from the group consisting of O, NH and N(C₁-C₆ alkyl);

R¹² is a member selected from the group consisting of H and C₁-C₆ alkyl, and when two or more R¹²'s are present in a single compound, such R¹²'s are the same or different;

p is 1, 2, or 3; and

q is 1 or 2; and

R⁷, when not combined with any R⁸, is a member selected from the group consisting of H, C₁-C₆ alkyl, and C₁-C₆ alkoxy.

A further class of compounds useful in the practice of the invention is those of Formula II:

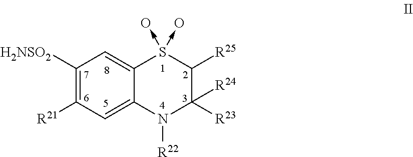

In Formula II:

R²¹ is either H, halo or CF₃

R²² and R²³ either are both H or are combined to form a double bond bridging the 3 and 4 ring vertices;

R²⁴ is either H, C₁-C₆ alkyl, C₅-C₇ cycloalkyl, C₅-C₇ cycloalkenyl, Ph, CH₂Ph, CH₂SCH₂Ph, CH₂X, CHX₂, CH₂SCH₂CF₃, CH₂SCH₂CH—CH₂, or

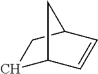

and R²⁵ is a member selected from the group consisting of H and C₁-C₆ alkyl.

Compounds 1 through 25 below are examples of compounds within the scope of Formula I:

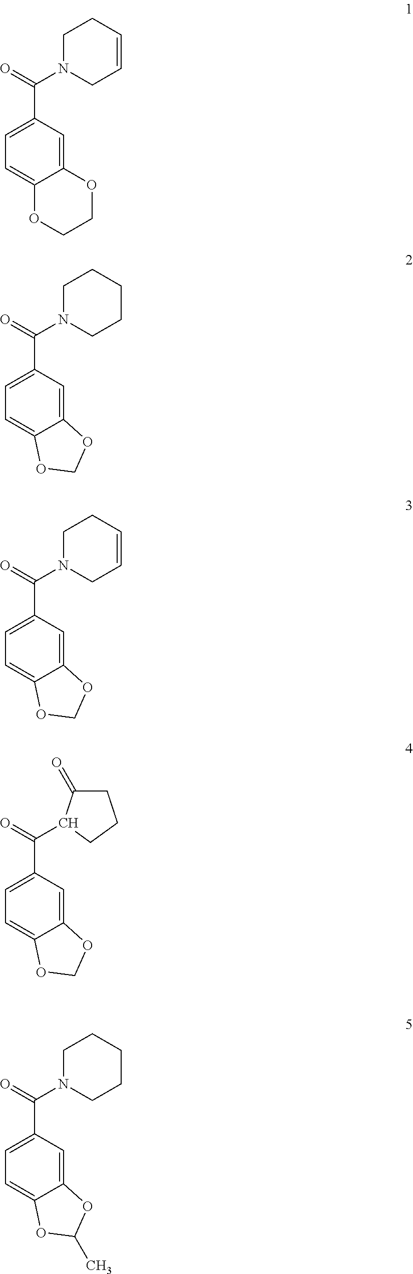

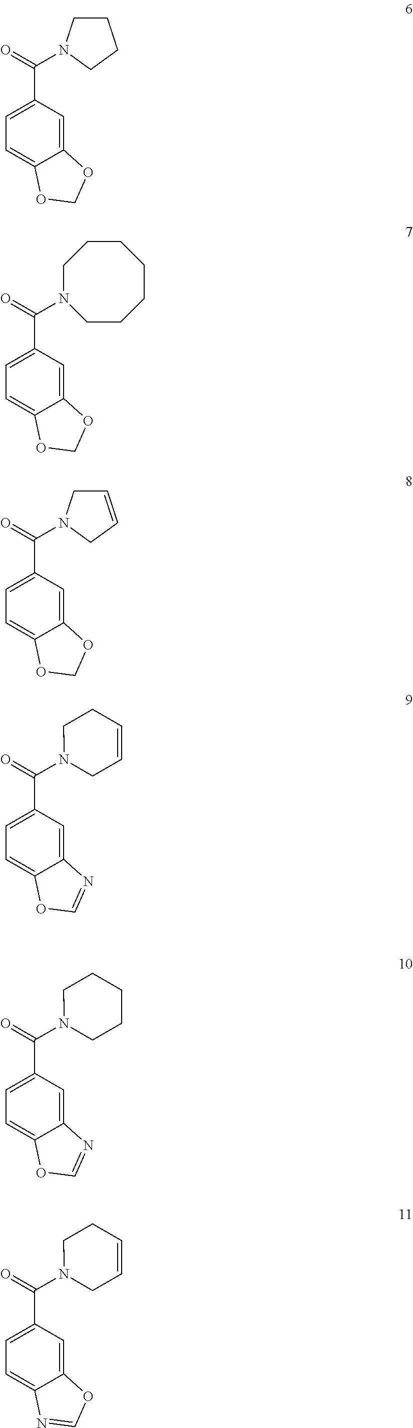

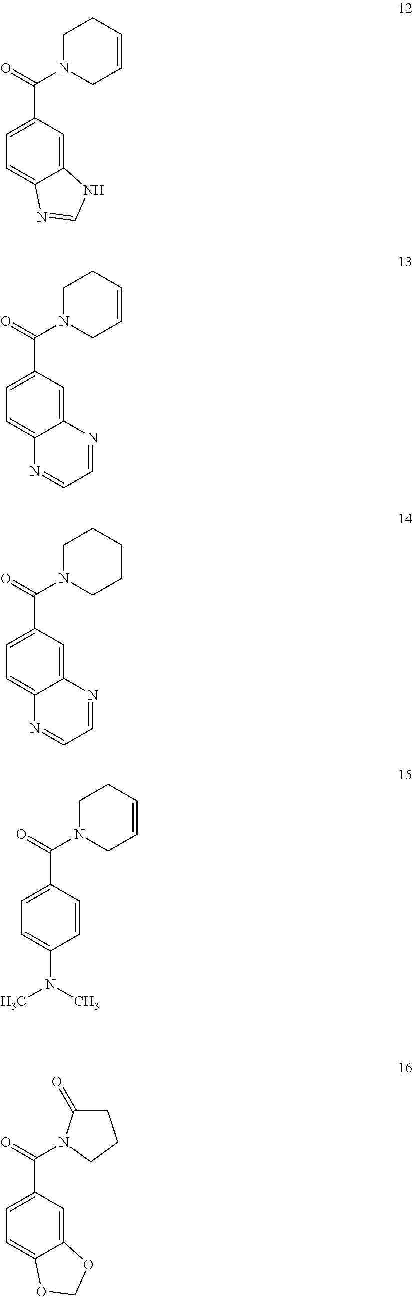 [SMILES: O=C(c1ccc2nc[nH]c2c1)N1CC=CCC1 ; O=C(c1ccc2nccnc2c1)N1CC=CCC1 ; O=C(c1ccc2nccnc2c1)N1CCCCC1 ; CN(C)c1ccc(C(=O)N2CC=CCC2)cc1 ; O=C1CCCN1C(=O)c1ccc2c(c1)OCO2]

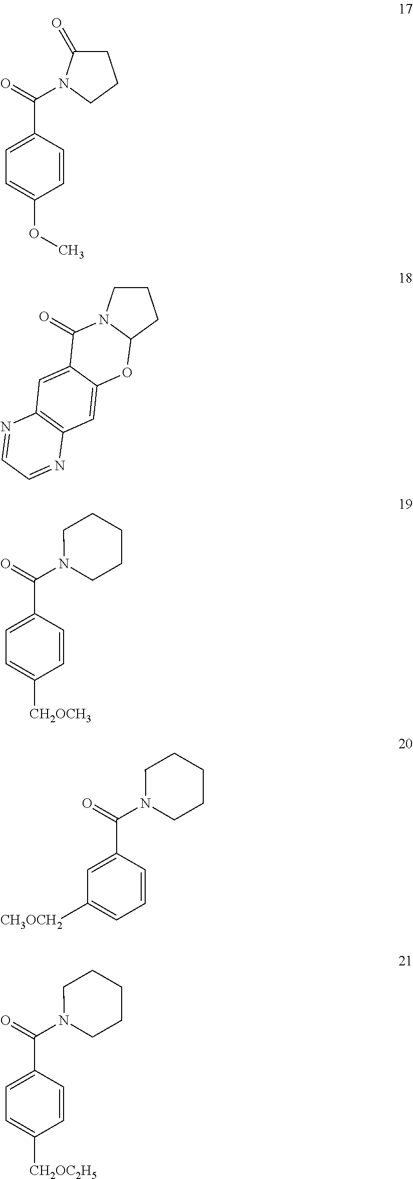

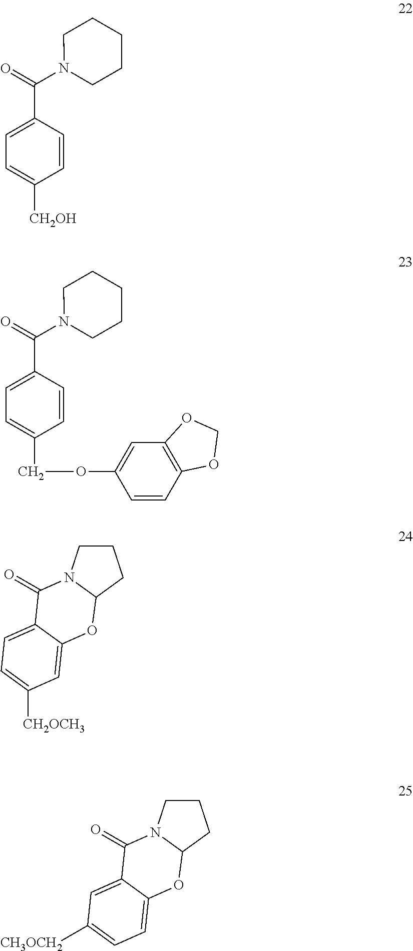

Compounds 26 through 40 below are compounds within the scope of Formula II:

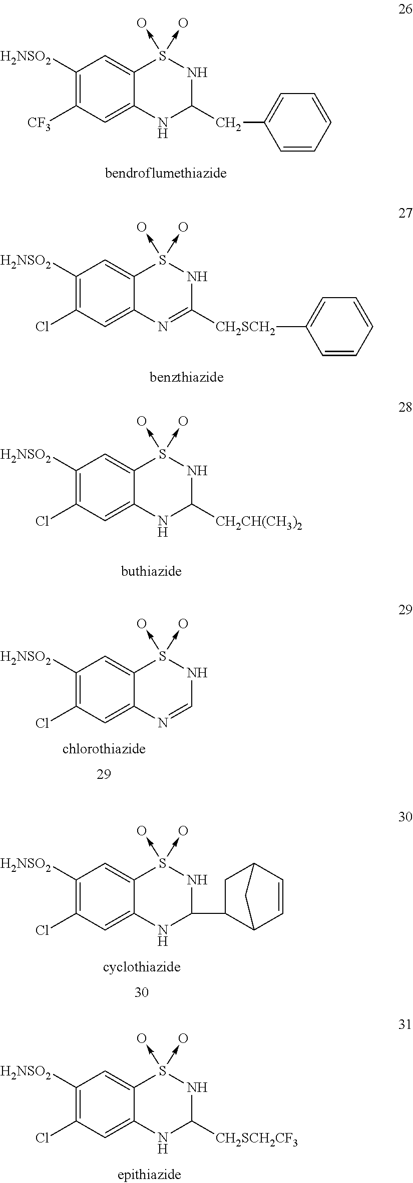

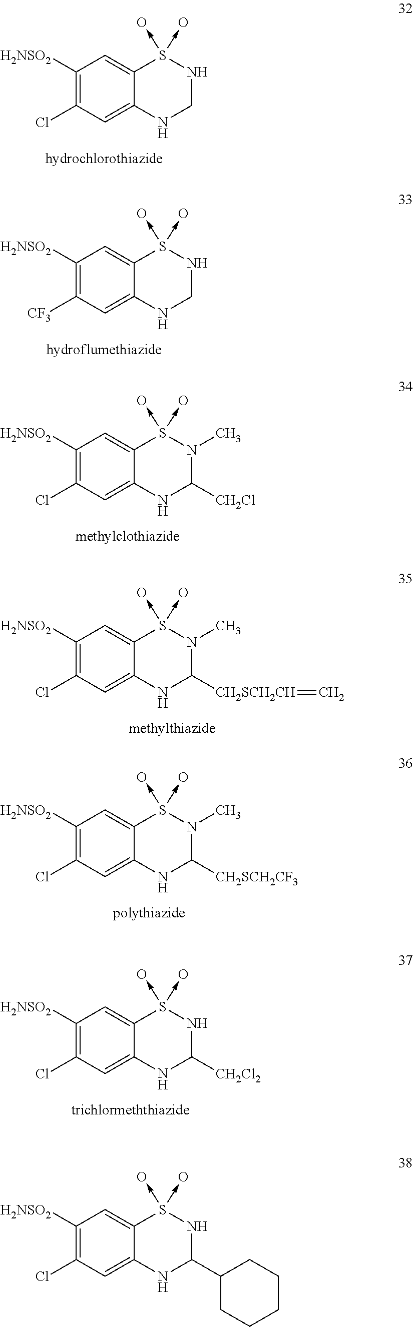

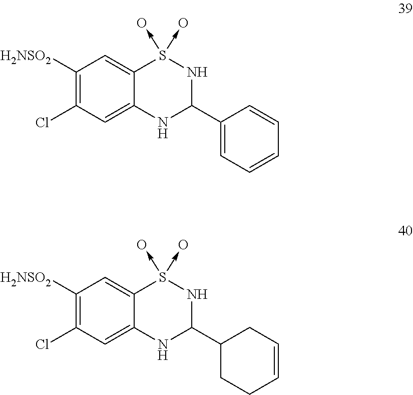

A particularly preferred compound is 1-(Quinoxalin-6-ylcarbonyl)piperidine, having the following structure:

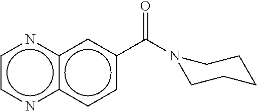

Another particularly preferred compound is 1-(1,4-benzodioxan-6-ylcarbonyl)piperidine, having the following structure:

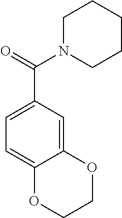 [SMILES: O=C(c1ccc2c(c1)OCCO2)N1CCCCC1]

In another embodiment, the ampakine is a compound of formula III:

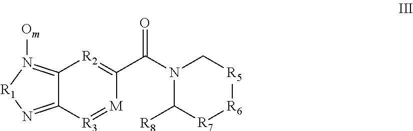

in which:

R¹ is oxygen or sulfur;

R² and R³ are independently selected from the group consisting of —N═, —CR═, and —CX═;

M is ═N or ═CR⁴—, wherein R⁴ and R⁸ are independently R or together form a single linking moiety linking M to the ring vertex 2′, the linking moiety being selected from the group consisting of a single bond, —CR₂—, —CR═CR—, —C(O)—, —O—, —S(O), —, —NR—, and —N═;

R⁵ and R⁷ are independently selected from the group consisting of —(C₂)ₙ—, —C(O)—, —CR═CR—, —CR═CX—, —C(RX)—, —CX₂—, —S—, and —O—; and

R⁶ is selected from the group consisting of —(CR₂)ₘ—, —C(O)—, —CR═CR—C(RX)—, —CR₂—, —S—, and —O—;

Wherein

X is —Br, —Cl, —F, —CN, —NO₂, —OR, —SR, —NR₂, —C(O)R—, —CO₂R, or —CONR₂; and

R is hydrogen, C₁-C₆ branched or unbranched alkyl, which may be unsubstituted or substituted with one or more functionalities defined above as X, or aryl, which may be unsubstituted or substituted with one or more functionalities defined above as X;

m and p are independently 0 or 1;

n and y are independently 0, 1 or 2.

Examples of compounds include:

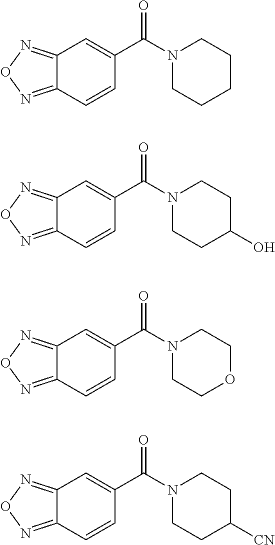 [SMILES: O=C(c1ccc2nonc2c1)N1CCCCC1 ; O=C(c1ccc2nonc2c1)N1CCC(O)CC1 ; O=C(c1ccc2nonc2c1)N1CCOCC1 ; N#CC1CCN(C(=O)c2ccc3nonc3c2)CC1]

### Preparation of Formula I Compounds

The compounds described above are prepared by conventional methods known to those skilled in the art of synthetic organic chemistry. For example, certain compounds of Formula I are prepared from an appropriately substituted benzoic acid by contacting the acid under conditions suitable to activate the carboxy group for the formation of an amide. This is accomplished, for example, by activating the acid with carbonyl diimidazole, or with a chlorinating agent such as thionyl chloride or oxalyl chloride to obtain the corresponding benzoyl chloride. The activated acid is then contacted with a nitrogen-containing heterocyclic compound under conditions suitable for producing the desired imide or amide. Alternatively, the substituted benzoic acid is ionized by contact with at least two equivalents of base such as triethylamine in an inert solvent such as methylene chloride or alcohol-free chloroform, and the ionized benzoic acid can then be reacted with pivaloyl chloride or a reactive carboxylic acid anhydride such as trifluoroacetic anhydride or trichloroacetic anhydride, to produce a mixed anhydride. The mixed anhydride is then contacted with a nitrogen-containing heterocyclic compound to produce the desired imide or amide.

A further alternative to these methods, suitable for some of the compounds of Formula I, is to contact the appropriately selected 3,4-(alkylenedihetero)-benzaldehyde with ammonia to form an imine, then contacting the imine with benzoyloxycarbonyl chloride to form the benzoyloxycarbonyl imine. Suitable 3,4-(alkylenedihetero)-benzaldehydes include 3,4-(methylenedioxy)-benzaldehyde, 3,4-(ethylenedioxy)-benzaldehyde, 3,4-(propylenedioxy)-benzaldehyde, 3,4-(ethylidenedioxy)-benzaldehyde, 3,4-(propylenedithio)-benzaldehyde, 3,4-(ethylidenedithio)-benzaldehyde, 5-benzimidazolecarboxaldehyde, and 6-quinoxalinecarboxaldehyde. The benzoyloxycarbonyl imine is then contacted with a simple conjugated diene such as butadiene under cycloaddition reaction conditions, and then with a Lewis acid under conditions suitable for a Friedel-Crafts acylation. Examples of suitable conjugated dienes include butadiene, 1,3-pentadiene, and isoprene, and examples of suitable Lewis acids include AlCl₃ and ZnCl₂.

Still further compounds within Formula I are prepared from 2,3-dihydroxy naphthalene. This starting material is reacted with 1,2-dibromoethane in the presence of base to produce an ethylenedioxy derivative of naphthalene, which is then reacted with an oxidizing agent such as potassium permanganate to produce 4,5-ethylenedioxyphthaldehydic acid. The latter is contacted with anhydrous ammonia to form an imine, which is then treated with a suitable carbonyl-activating agent such as dicyclohexylcarbodiimide under cyclization conditions to form an acyl imine. The acyl imine is then reacted with a simple conjugated diene to achieve cycloaddition.

Still further compounds within Formula I are prepared by contacting an α-halotoluic acid with at least two equivalents of an alkali salt of a lower alcohol according to the Williamson ether synthesis to produce an ether linkage. The resulting alkoxymethylbenzoic acid is activated with carbonyldiimidazole, thionyl chloride, dicyclohexylcarbodiimide, or any other suitable activating agent, and reacted with a suitable amine to achieve a carboxamide linkage.

In an alternate to the scheme of the preceding paragraph, a formyl-substituted aromatic carboxamide is prepared by activation of an appropriate starting acid with a tertiary amine (for example, triethyl amine) plus an acid chloride (for example, pivaloyl chloride) to produce a mixed anhydride for coupling to a suitable amine. The formyl group is then reduced to an alcohol by a suitable reducing agent such as sodium borohydride. The alcohol is then converted to a leaving group which is replaceable by the alkali salt of an alcohol. The leaving group can be generated by reagents such as thionyl chloride, thionyl bromide, mineral acids such as hydrochloric, hydrobromic or hydroiodic acids, or the combined action of a tertiary amine plus either a suitable sulfonic anhydride or sulfonyl halide. Alternatively, the alcohol is activated by removing the proton. This is achieved by the action of a strong base such as sodium hydride in an aprotic solvent such as dimethylformamide. The resulting alkoxide is then reacted with a suitable alkyl halide or other alkyl compound with a suitable leaving group to produce the desired ether linkage.

Fused ring structures such as those in which R³ and one of the R⁸'s of Formula I are combined to form a single linking group bridging the 2 and 3′ carbon atoms can be synthesized in the following manner. The carboxyl group of an appropriately substituted salicylic acid is activated with carbonyldiimidazole in dichloromethane, chloroform, tetrahydrofuran, or other anhydrous solvent. An aminoalkylacetal such as H₂N(CH₂)₃CH(OCH₂CH₃)₂ is then added. The resulting amide is treated with an aryl or alkyl sulfonic acid, trifluoroacetic acid, or other strong acid, in a solvent of low basicity such as chloroform or dichloromethane, to cleave the acetal and cyclize the intermediate aldehyde with the amide nitrogen and the phenolic oxygen.

In all of these reaction schemes, the methods and reaction conditions for each of the individual reactions are well within the routine skill of, and will be readily apparent to, the synthesis chemist.

The above described genus and species of compounds represent merely one example of ampakines that may be used to treat non-neurodegenerative pathologies associated with according to the present invention. The treatments provided by present invention are not limited to the compounds described above. The present invention also encompasses administering other compounds that enhance the stimulation of α-amino-3-hydroxy-5-methyl-isoxazole-4-propionic acid (“AMPA”) receptors in a subject and BDNF experssion in the brain stem. Examples of other such AMPA-selective compounds include 7-chloro-3-methyl-3-4-dihydro-2H-1,2,4 benzothiadiazine S,S, dioxide, as described in Zivkovic et al., 1995, J. Pharmacol. Exp. Therap., 272:300-309; Thompson et al., 1995, Proc. Nat. Acad. Sci. USA, 92:7667-7671, all of which are herein incorporated by reference in their entirety.

In still further embodiments, the present invention provides methods for the use of a pharmaceutical composition suitable for administering an effective amount of at least one ampakine, such as those disclosed herein, in unit dosage form to treat non-neurodegenerative pathologies associated with deranged or impaired BDNF signaling. In alternative embodiments, the composition further comprises a pharmaceutically acceptable carrier.

The therapeutic agents of the present invention (e.g., the compounds in Formulas I-III and the others described above) are capable of further forming both pharmaceutically acceptable acid addition and/or base salts. All of these forms are within the scope of the present invention and can be administered to the subject to treat non-neurodegenerative pathologies associated with deranged or impaired BDNF signaling.

Pharmaceutically acceptable acid addition salts of the present invention include, but are not limited to, salts derived from nontoxic inorganic acids such as hydrochloric, nitric, phospohoric, sulfuric, hydrobromic, hydriodic, hydrofluoric, phosphorous, and the like, as well as the salts derived forth nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bissulfite, nitrate, phosphate, monoLydrogenphosphate, dihydrogenphosphate, metaphosphate, pyrophosphate, chloride, bromide, iodide, acetate, trifluoracetate, propionate, caprylate, isobutyrate, oxalate, malonate, succinate, suberate, sebacate, fumarate, malcate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like, as well as gluconate, galacturonate, and n-methyl glucamine.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner or as described above. The free base forms differ from their respective salt forms somewhat in certain physical properties such as solubility in polar solvents, but are otherwise equivalent to their respective free base for purposes of the present invention.

Pharmaceutically acceptable base addition salts are formed with metals or amides, such as alkali and alkaline earth metals or organic amines. Examples of metals used as cations include, but are not limited to, sodium, potassium, magnesium, calcium, and the like. Examples of suitable amines include, but are not limited to, N2,N′-dibenzylethylenediamine, chloroprocaine, choline, diethanolamine, dicyclohexylamine, ethylenediamine, N-methylglucamine, and procaine.

The base addition salts of the acidic compounds are prepared by contacting the free acid form with a sufficient amount of the desired base to produce the salt in the conventional manner. The free acid form may be regenerated by contacting the salt form with an acid and isolating the free acid in the conventional manner or as described above. The free acid forms differ from their respective salt forms somewhat in certain physical properties such as solubility in polar solvents, but otherwise the salts are equivalent to their respective free acid for purposes of the present invention.

Certain compositions of the present invention can exist in unsolvated forms as well as solvated forms, including, but not limited to, hydrated forms In general, the solvated forms, including hydrated forms, are equivalent to unsolvated forms and are intended to be encompassed within the scope of the present invention. Certain of the compounds of the present invention possess one or more chiral centers and each center may exist in different configurations. The compounds can, therefore, form stereoisomers. Although these are all represented herein by a limited number of molecular formulas, the present invention includes the use of both the individual, isolated isomers and mixtures, including racemates, thereof. Where stereospecific synthesis techniques are employed or optically active compounds are employed as starting materials in the preparation of the compounds, individual isomers may be prepared directly. However, if a mixture of isomers is prepared, the individual isomers may be obtained by conventional resolution techniques, or the mixture may be used as is, with resolution.

For preparing pharmaceutical compositions from the compounds of the present invention, pharmaceutically acceptable carriers can be in any suitable form (e.g., solids, liquids, gels, aerosols, etc.). Solid form preparations include, but are not limited to, powders, tablets, pills, capsules, cachets, suppositories, and dispersible granules. A solid carrier can be one or more substances which may also act as diluents, flavoring agents, binders, preservatives, tablet disintegrating agents, or an encapsulating material. The present invention contemplates a variety of techniques for administration of the therapeutic compositions. Suitable routes include, but are not limited to, oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, among others. Indeed, it is not intended that the present invention be limited to any particular administration route.

For injections, the agents of the present invention may be formulated in aqueous solutions, preferably in physiologically compatible buffers such as Hank's solution, Ringer's solution, or physiological saline buffer. For such transmucosal administration, penetrants appropriate to the barrier to be permeated are used in the formulation. Such penetrants are generally known in the art.

In powders, the carrier is a finely divided solid which is in a mixture with the finely dived active component. In tablets, the active component is mixed with the carrier having the necessary binding properties in suitable proportions, which has been shaped into the size and shape desired.

The powders and tablets preferably contain from five or ten to about seventy percent of the active compounds. Suitable carriers include, but are not limited to, magnesium carbonate, magnesium stearate, talc, sugar, lactose, pectin, dextrin, starch, gelatin, tragacanth, methylcellulose, sodium carboxymethylcellulose, a low melting wax, cocoa butter and the like, among other embodiments (e.g., solid, gel, and liquid forms). The term “preparation” is intended to also encompass the formation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, in some embodiments of the present invention, a low melting wax, such as a mixture of fatty acid glycerides or cocoa butter; is first melted and the active compound is dispersed homogeneously therein, as by stirring. The molten homogenous mixture is then poured into convenient sized molds, allowed to cool, and thereby to solidify in a form suitable for administration

Liquid form preparations include, but are not limited to, solutions, suspensions, and emulsions (e.g., water or water propylene glycol solutions). For parenteral injection, in some embodiments of the present invention, liquid preparations are formulated in solution in aqueous polyethylene glycol solution. Aqueous solutions suitable for oral use can be prepared by dissolving the active component in water and adding suitable colorants, flavors, and stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing appropriate quantities of the active component. The unit dosage form can be a packaged preparation, the package containing discrete quantities of preparation, such as tablets, capsules, and powders in vials or ampoules. Also, the unit dosage form can be a capsule, tablet, cachet, or lozenge itself, or it can be the appropriate number of any of these in packaged form.

General procedures for preparing pharmaceutical compositions are described in Remington's Pharmaceutical Sciences, E. W. Martin, Mack Publishing Co., PA (1990), which is herein incorporated by reference in it entirety.

In one aspect of the invention the amount of ampakine administered to a subject can be that amount effective to enhance brain-derived neurotrophic factor (BDNF) expression from NG neurons and brain stem neurons in brain stem of the subject and improve respiratory and neurological function of the subject. The quantity of active component in a unit dose preparation may be varied or adjusted from 0.1 mg/kg per day to about 100 mg/kg per day, for example, ranging from 10 mg/kg per day to about 50 mg/kg per day according to the particular application and the potency of the active component. The composition can, if desired, also contain other compatible therapeutic agents.

The assessment of the clinical features and the design of an appropriate therapeutic regimen for the individual patient is ultimately the responsibility of the prescribing physician. It is contemplated that, as part of their patient evaluations, the attending physicians know how to and when to terminate, interrupt, or adjust administration due to toxicity, or to organ dysfunctions. Conversely, the attending physicians also know to adjust treatment to higher levels, in circumstances where the clinical response is inadequate, while precluding toxicity. The magnitude of an administrated dose in the management of the disorder of interest will vary with the severity of the condition to be treated, the patient's individual physiology, biochemistry, etc., and to the route of administration. The severity of the condition, may, for example, be evaluated, in part, by standard prognostic evaluation methods.

Further, the dose and dose frequency will also vary according to the age, body weight, sex and response of the individual patient.

The following example is provided in order to demonstrate and further illustrate certain preferred embodiments and aspects of the present invention and is not to be construed as limiting the scope thereof.

### Example

### Ampakine Treatment Enhances BDNF Levels and Respiratory Function in a Mouse Model of Rett Syndrome.

We examined BDNF expression in nodose cranial sensory ganglia (NG) neurons cultured under depolarizing and non-depolarizing conditions to test the hypothesis that that decreased neuronal activity in Mecp2 null mutants reduces activity-dependent BDNF expression. Because the NG is comprised of a single neuronal cell type, sensory neurons, and exhibits the Mccp2 null BDNF phenotype in vitro as in vivo, it provides a simple model for exploring mechanisms that underlie BDNF regulation by McCP2. Our data indicate that Mecp2 null cells exhibit significantly lower levels of BDNF expression than wildtype, regardless of their activity state. However, BDNF levels in mutant cells can be elevated to wildtype resting levels by depolarizing stimuli in vitro. Similarly, we find that treatment of Mecp2 null mice with the ampakine drug CX546, which enhances activation of glutamatergic AMPA receptors, elevates NG BDNF levels in vivo. Moreover, ampakine treatment significantly improves respiratory function in Mecp2 null mice, suggesting that this class of compounds may be of therapeutic value in the treatment of Rett's syndrome (RTT).

### Methods

### Animals

Mecp^{2tml-1Jae} mice (Chen et al., 2001), developed by Dr. R. Jaenisch (Whitehead Institute, MIT) and obtained from the Mutant Mouse Regional Resource Center (MMRRC, UC Davis, CA), were maintained on a mixed background. Male Mecp2 nulls (Mecp2^{-/y}) were generated by crossing heterozygous Mecp2^{tml-Jae} knock-out females with Mecp2^{tml-Jae} wildtype males (Mecp2^{-/y}). All experimental procedures were approved by the Institutional Animal Care and Use Committee at Case Western Reserve University.

### Cell Cultures

Wildtype and Mecp2 null mice were killed with CO₂ on postnatal day 35 (P35). The NGs were removed, digested in 0.1% collagenase (Sigma, St. Louis, Mo.) in Earle's balanced salt solution (Invitrogen, San Diego, Calif.) for 70 min. at 37° C., triturated in culture medium (see below) containing 0.15% BSA, and plated at a density of one NG per well into 96-well flat-bottom ELISA plates coated with poly-D-lysine. Cultures were grown for 3 d in DMEM-F-12 medium supplemented with 5% fetal bovine serum (Invitrogen) and 1% penicillin-streptomycin-neomycin, with or without 40 mM KCl or 1.5 μM tetrodotoxin.

### Ampakine Treatment

Beginning on P25, wildtype and Mecp2 null littermates were acclimatized to the injection protocol to reduce stress, first by handling for 10 min./day for 3 days, followed by saline injections (0.9% NaCl, i.p., b.i.d.) at 8:00 AM and 8:00 PM, for an additional 3 days. Subsequently, mice were assigned either to drug treatment (CX546, 40 mg/kg in 16.5% 2-hydroxypropyl-3-cyclodextrin, i.p., b.i.d.) or vehicle injections (cyclodextrin alone). On the day of their last injection, mice were trained in the plethysmograph recording chamber for one hour. Eighteen to 24 hours after their last injection, on P35, mice were returned to the chamber for recording of respiratory activity.

### Plethysniography

Breathing was recorded in unrestrained mice using a whole-body flow plethysmograph (Buxco II; Buxco Research Systems, Wilmington, N.C.) in which a constant bias flow supply connected to the animal recording chamber ensured continuous inflow of fresh air (1 L/min.). Ambient temperature was maintained between 23 and 25° C. Breathing traces were analyzed using Biosystem XA software (Buxco Research Systems). After the recording sessions, mice were euthanized with CO₂ and tissue processed for BDNF immunoassay.

### BDNF Immunoassay

BDNF protein levels in intact NG or in cultured NG cells were measured by ELISA using the BDNF Emax Immunoassay System (Promega). Protein extracts from one intact NG or from an equivalent number of cultured cells were used for ELISA.

### MeCP2 and β3-Tubulin Doable-Staining

Mice were killed with CO₂, perfused with 4% paraformaldehyde, and the head sectioned at 10 μm with a cryostat. Sections were stained with rabbit polyclonal anti-MeCP2 (Upstate, Lake Placid, N.Y.) and chicken polyclonal anti-β3-tubulin (Ayes labs, Ft. Lauderdale, Fla.).

### Statistical Analysis

Differences between wildtype and mutant mice, and between vehicle-treated and CX546-treated mice, were tested using unpaired t test or ANOVA I with Tukey's multiple comparison post-hoc analysis. A p value <0.05 was considered statistically significant. Data are presented as mean±SEM.

### Results

### A Cell-Autonomous BDNF Deficit in Mecp2 Null Mice

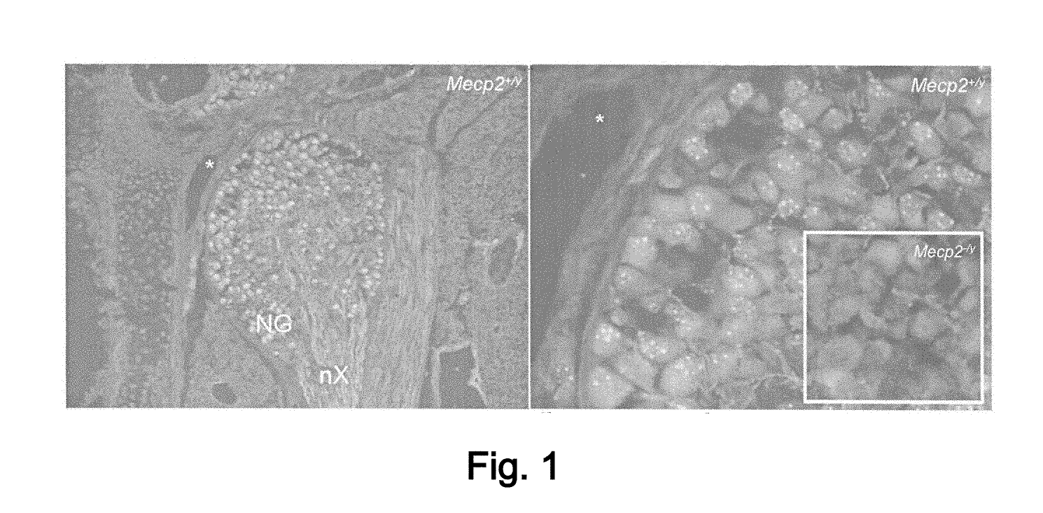

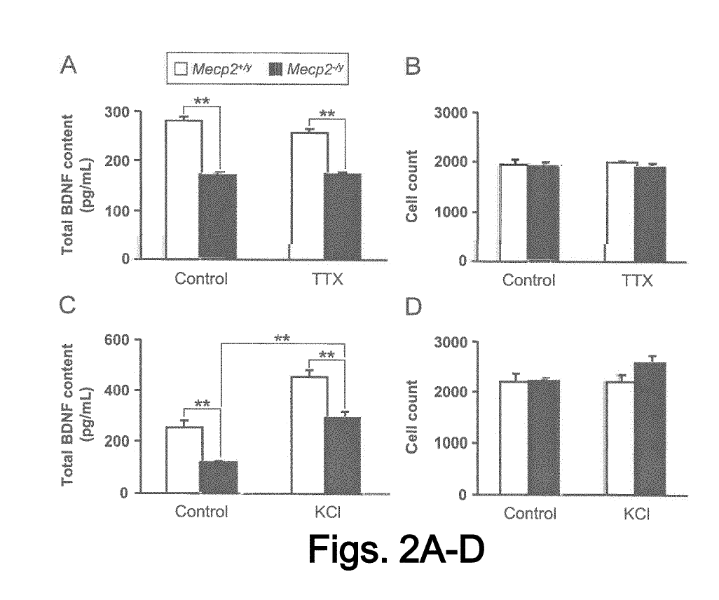

The marked deficit in BDNF content found in adult Mecp2 null NG neurons in vivo is maintained in dissociate cell culture, suggesting that it is a cell-autonomous effect of MeCP2 loss. However, MeCP2 expression in peripheral neurons has not previously been described. Therefore, initial studies examined the localization of MeCP2 immunoreactivity in the NG and found robust expression in all neurons at PO through P35 (FIG. 1). To test the hypothesis that differences in BDNF content between wildtype and Mecp2 null cells result from different levels of activity, BDNF levels were compared in P35 NG neurons from wildtype and Mecp2 null mice grown in dissociated culture for 3 days under control (non-depolarizing) and depolarizing (40 mM KCl) conditions. Under control conditions, NG neurons exhibit resting membrane potentials of approximately −70 mV and are not spontaneously active. However, to eliminate any possible depolarizing influence of voltage-gated sodium channels, some cultures were grown in the presence of 1.5 μM tetrodotoxin (TTX; NG neurons also express TTX-insensitive Na channels, however, these activate at substantially more positive membrane potentials. In both control and TTX treated cultures, Mecp2 null neurons exhibit 40-50% less BDNF than wildtype neurons (FIG. 2A), as in vivo, without any change in cell survival (FIG. 2B).

To further test the role of membrane depolarization in the BDNF phenotype of Mecp2 null neurons, NG cultures were grown in the absence and presence of a depolarizing concentration of potassium chloride (KCl; 40 mM). In both wildtype and mutant cultures, KCl depolarization resulted in a significant increase in BDNF protein compared to unstimulated controls (FIG. 2C), with no change in cell survival (FIG. 2D). However, even under depolarizing conditions, mutant cells expressed significantly lower levels of BDNF than wildtype cells. These data indicate that Mecp2 is required for normal levels of BDNF expression in NG neurons under both resting and depolarizing conditions. In addition, these experiments show that chronic depolarization of mutant neurons can stimulate BDNF protein expression to wildtype resting levels.

### Ampakine Stimulation of BDNF Expression In Vivo

The fact that depolarization of Mecp2 null NG neurons could increase BDNF expression in vitro raised the possibility that neuronal activation could rescue the BDNF deficit in vivo. To approach this issue we examined the effect of an ampakine drug, CX546, on BDNF protein expression in the NG in intact P35 wildtype and Mecp2 null mice. Ampakines are fast-acting molecules that acutely lengthen the duration of AMPA receptor-mediated inward currents and thereby increase the activity of neurons that express AMPA receptors. As a result, repeated ampakine treatment leads to an increase in activity-dependent expression of BDNF, in vivo and in vitro.

Wildtype and Mecp2 null littermates were treated for 3 days with CX546 (40 mg/kg in cyclodextrin, i.p., b.i.d.) or vehicle. Respiratory function was monitored by whole-body plethysmography 18-24 hours after the last injection, as described in Methods. At the end of the recording session the mice were sacrificed and the NG removed for BDNF ELISA. NG BDNF content in vehicle-treated Mecp2 null mice was significantly reduced compared to wildtype controls, as previously described in naïve untreated animals (wildtype vs. mutant, 170±14 vs. 72±3 pg BDNF/mL, n=6, p<0.001, ANOVA I). Treatment of wildtype mice with CX546 had no effect on NO BDNF content. However, treatment of Mecp2 null mice resulted in a significant 42% increase in BDNF protein content compared to vehicle-treated mutants (wildtype CX546 vs. mutant CX546, 167±5 vs. 114±4 pg BDNF/mL, n=6, p<0.001, ANOVA I).

Ampakine Treatment Restores Wildtype Mean Respiratory Frequency and Minute volume in Mecp2^{tml-1Jae} null mice

NG neurons secrete BDNF in an activity-dependent manner and BDNF acutely modulates glutamatergic transmission at second-order neurons in the nucleus tractus solitarius (nTS), the primary relay for peripheral afferent input to the brainstem respiratory rhythm generating network. Therefore, we hypothesize that BDNF deficits in NG neurons contribute to the pathogenesis of respiratory dysfunction in RTT by disrupting synaptic modulation in nTS.

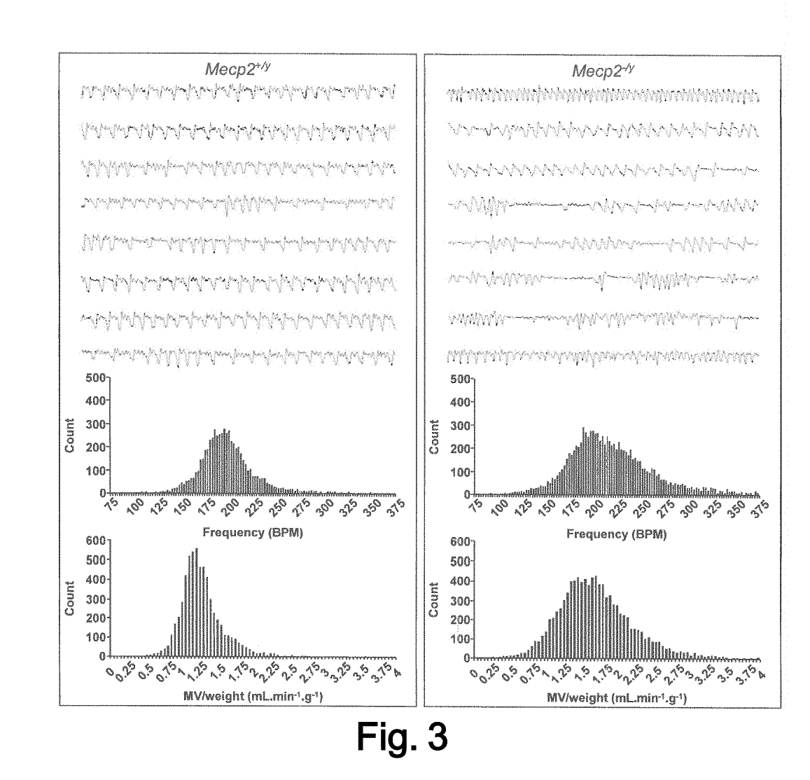

To examine whether or not ampakine enhancement of BDNF expression in Mecp2 null NG neurons is associated with recovery of neural function, we compared respiratory activity in wildtype and mutant mice following treatment with CX546 in vivo using whole-body plethysmography. Analysis of naïve untreated wildtype and mutant animals revealed a highly disordered breathing pattern in the mutants compared to wildtype controls (FIG. 3). The mutant breathing pattern is characterized by a highly variable frequency (Coefficient of variation of breathing frequency, wildtype vs. mutant, 18.8±0.7 vs. 22.0±1.2%, n=6 for wildtype and n=7 for mutants, p<0.05, unpaired t test) and occasional long breathing pauses compared to wildtypes, similar to human RTT patients and other mouse models (Mecp2^{tml-1Bird} null mice). More detailed analysis of breathing parameters revealed that the phenotype observed in mutant mice is associated with repetitive episodes of very high breathing frequency (FIG. 3), resulting in a 23% increase in mean respiratory frequency compared to wildtype controls (p<0.001, n=6 for wildtype and n=7 for mutants, unpaired t test). Consequently, the mean value for minute volume/weight is also increased in mutants (FIG. 3; wildtype vs. mutant, 0.97±0.11 vs. 1.38±0.13 mL/min./g., n=6 for wildtypes and n=7 for mutants, p<0.05, unpaired t test). In contrast, there was no significant difference in tidal volume/weight between wildtype and mutant animals (wildtype vs. mutant, 5.4±0.6 vs. 6.4±0.5 n6 for wildtypes and n=7 for mutants).

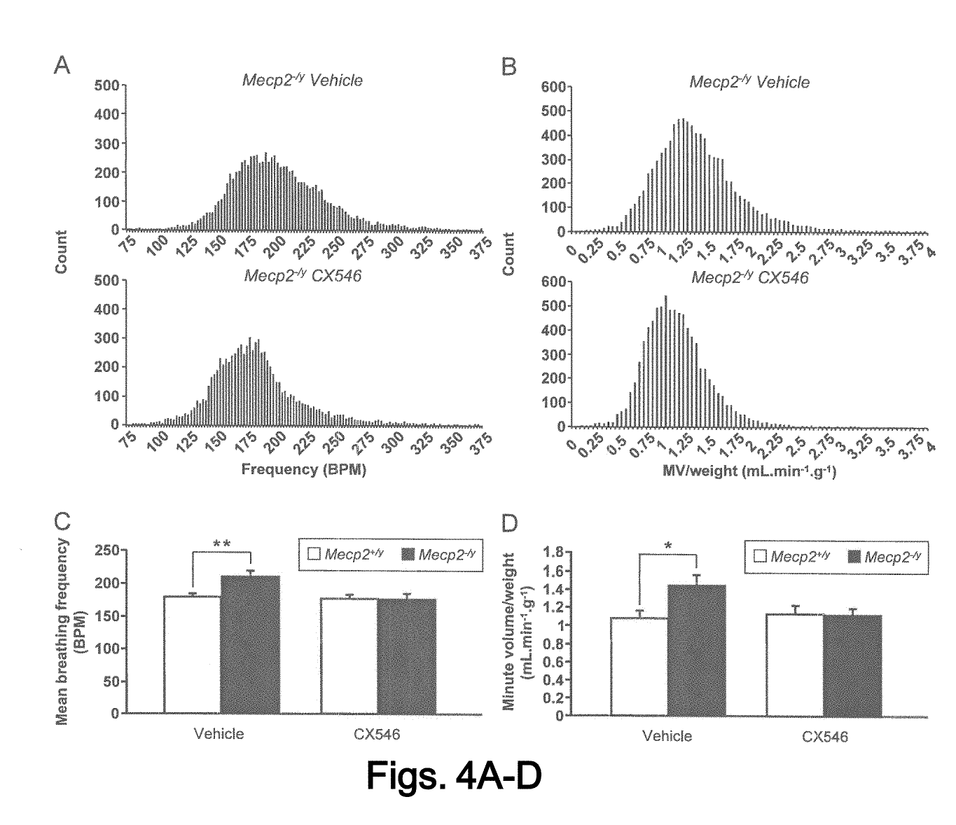

Three-day treatment with CX546 did not significantly affect breathing frequency, tidal volume/weight and minute volume/weight in P35 Mecp2^{tml-Jae} wildtype mice (vehicle vs. CX546, frequency: 179±3 vs. 177±6 breath/min., tidal volume/weight: 6.1±0.4 vs. 6.4±0.5 μL/g., minute volume/weight: 1.09±0.07 vs. 1.14±0.09 mL/min./g., n=8 for vehicle and n=7 for CX546). In contrast, ampakine treatment of mutant animals sharply decreased the episodes of high breathing frequency, leading to restoration of wildtype mean breathing frequency (FIG. 4A,C; wildtype CX546 vs. mutant CX546, 177±6 vs. 176±8 breath/min., n=7 for wildtypes and n=9 for mutants) and minute volume/weight (FIG. 4B,D; wildtype CX546 vs. mutant CX546, 1.14±0.09 vs. 1.13±0.07 mL/min./g., n=7 for wildtypes and n=9 for mutants). However, ampakine treatment did not decrease the higher variability in breathing frequency characteristic of mutant animals (Coefficient of variation of breathing frequency, wildtype CX546 vs. mutant CX546, 18.5±1.2 vs. 23.4±1.5%, n=7 for wildtypes and n=9 for mutants). Tidal volume/weight was not affected in mutants by ampakine treatment and was similar to wildtype (wildtype CX546 vs. mutant CX546, 6.4±0.5 vs. 6.5±0.3 μL/g., n=7 for wildtypes and n=9 for mutants).

### DISCUSSION

Our results demonstrate that MeCP2 is required for normal levels of BDNF expression in nodose sensory neurons under both resting and depolarizing conditions in vitro. Moreover, chronic depolarization in vitro, or ampakine treatment in vivo, can elevate BDNF levels in Mecp2 null cells. Furthermore, ampakine treatment results in a restoration of wildtype breathing frequency and minute volume/weight in Mecp2 null mice.

Previous studies in cultured newborn cortical neurons indicated that MeCP2 represses BDNF expression at rest and that release from MeCP2 mediated repression is required for activity dependent expression of BDNF. On the other hand, Mecp2 null mice exhibit BDNF deficits in vivo. One explanation for this discrepancy is that Mecp2 null cortical neurons are less active in vivo than wildtype cells, leading to a reduction in activity-dependent BDNF expression that masks any effects of BDNF depression. However, our data indicate that, as in vivo, Mecp2 null NG neurons express significantly less BDNF than wildtype cells when grown in dissociated cell culture, under both non-depolarizing and depolarizing conditions. Thus, the BDNF deficit in these cells appears to be independent of the state of depolarization. This apparent difference in BDNF regulation in Mecp2 null cortical and NG neurons, respectively, may indicate a role for cell context in determining the interaction between these two genes. We cannot rule out the possibility that, in NG neurons, MeCP2 indirectly regulates BDNF expression, perhaps by repressing a gene or genes that, in turn, repress BDNF.

The fact that BDNF expression remains plastic in Mecp2 null NG neurons and can be increased by depolarizing stimuli in vitro led us to test whether or not BDNF levels could be increased in Mecp2 null mice in vivo by the ampakine drug CX546. Ampakines are a family of small molecules that trigger short-term increases in the duration of AMPA-mediated inward currents. In addition, repeated treatment with ampakines can increase the efficiency of long-term potentiation in the hippocampus and facilitate memory processes. These long term effects of ampakine treatment result from their ability to increase BDNF mRNA and protein expression.

Our study reveals that chronic treatment with CX546 significantly improves respiratory behavior in adult symptomatic Mecp2 null mice by decreasing breathing frequency and minute volume/weight. The respiratory improvement was not an acute effect of ampakine treatment, as CX546 has an extremely short half-life (less than an hour) and breathing was analyzed 18-24 hours after the last drug injection. Although mechanisms that underlie improved respiration in ampakine-treated Mecp2 null mice remain to be defined, our data are consistent with a role for increased BDNF expression in the NG. NG neurons project centrally to the brainstem nucleus tractus solitarius (nTS), the primary site for afferent input to the brainstem respiratory rhythm generating network, where BDNF inhibits glutamatergic excitation of second order vagal sensory relay neurons. In Mecp2 null mice, BDNF is severely depleted in NG afferents and their projections to nTS and activity of post-synaptic neurons is increased compared to wildtype controls. Thus, we suspect that elevated respiratory frequency in Mecp2 null mice may result in part from increased excitability in nTS and that ampakine treatment restores wildtype respiratory frequency by enhancing BDNF modulation of primary afferent transmission. This possibility is supported by recent findings that breathing dysfunction in Mecp2 null mice results from enhanced excitatory (or decreased inhibitory) neurotransmission affecting both vagal sensory and brainstem respiratory cell groups.

Neuropathological studies in RTT patients and Mecp2 null mice indicate relatively subtle structural abnormalities, such as decreased dendritic arbor complexity, that likely reflect disruptions in transynaptic signaling rather than overt neuronal degeneration, raising the possibility that functional deficits in RTT may be reversible. This possibility has recently been strengthened by the demonstration that postnatal re-expression of Mecp2 in severely symptomatic Mecp2 null mice is associated with symptom reversal. Our findings demonstrate that ampakine treatment of symptomatic Mecp2 null mice can significantly improve respiratory function, raising the possibility that this class of compounds may be of therapeutic value in the treatment of RTT patients.

1-19. (canceled)

20: A method of treating respiratory dysfunction in a subject caused by loss-of-function mutations of the gene encoding methyl-CpG binding protein 2 (MeCP2), comprising:
chronically administering to the subject an amount of at least one ampakine effective to increase brain-derived neurotrophic factor expression in brain stem neurons and nodose sensory neurons and improve respiratory function of the subject; and
measuring respiratory activity in the subject following administration of the at least one ampakine.

21: The method of claim 20, wherein the subject has Rett Syndrome.

22: The method of claim 20, the ampakine being an allosteric modulator of the AMPA-receptor.

23: The method of claim 20, the ampakine comprising a compound having the formula: 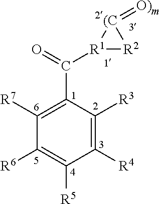
wherein,
R¹ is a member selected from the group consisting of N and CH;
m is 0 or 1;
R² is a member selected from the group consisting of (CR⁸₂)ₙ₋ₘ and Cₙ₋ₘR⁸₂₍ₙ₋ₘ₎₋₂, in which n is 4, 5, 6, or 7, the R⁸'s in any single compound being the same or different, each R⁸ being a member selected from the group consisting of H and C₁-C₆ alkyl, or one R⁸ being combined with either R³ or R⁷ to form a single bond linking the no. 3′ ring vertex to either the no. 2 or the no. 6 ring vertices or a single divalent linking moiety linking the no. 3′ ring vertex to either the no. 2 or the no. 6 ring vertices, the linking moiety being a member selected from the group consisting of CH₂, CH₂CH₂, CH═CH, O, NH, N(C₁-C₆ alkyl), N═CH, N═C(C₁-C₆ alkyl), C(O), O—C(O), C(O)—O, CH(OH), NH—C(O), and N(C₁-C₆ alkyl)-C(O);
R³, when not combined with any R⁸, is a member selected from the group consisting of H, C₁-C₆ alkyl, and C₁-C₆ alkoxy;
R⁴ is either combined with R⁵ or is a member selected from the group consisting of H, OH, and C₁-C₆ alkoxy;
R⁵ is either combined with R⁴ or is a member selected from the group consisting of H, OH, C₁-C₆ alkoxy, amino, mono(C₁-C₆ alkyl)amino, di(C₁-C₆ alkyl)amino, and CH₂OR⁹, in which R⁹ is a member selected from the group consisting of H, C₁-C₆ alkyl, an aromatic carbocyclic moiety, an aromatic heterocyclic moiety, an aromatic carbocyclic alkyl moiety, an aromatic heterocyclic alkyl moiety, and any such moiety substituted with one or more members selected from the group consisting of C₁-C₃ alkyl, C₁-C₃ alkoxy, hydroxy, halo, amino, alkylamino, dialkylamino, and methylenedioxy;
R⁶ is either H or CH₂OR⁹;
R⁴ and R⁵ when combined form a member selected from the group consisting of 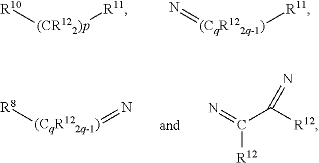
in which: R¹⁰ is a member selected from the group consisting of O, NH and N(C₁-C₆ alkyl);
R¹¹ is a member selected from the group consisting of O, NH and N(C₁-C₆ alkyl);
R¹² is a member selected from the group consisting of H and C₁-C₆ alkyl, and when two or more R¹²'S are present in a single compound, such R¹²'S are the same or different;
p is 1, 2, or 3; and
q is 1 or 2; and
R⁷, when not combined with any R⁸, is a member selected from the group consisting of H, C₁-C₆ alkyl, and C₁-C₆ alkoxy.

24: The method of claim 8, the ampakine comprising at least one of 1-(1,4-benzodioxan-6-ylcarbonyl)piperidine, 1-(quinoxalin-6-ylcarbonyl)piperidine, 2H,3H,6aH-pyrrolidino[2″,1″-3′,2″]1,3-oxazino[6′,5′-5,4]benzo[e]1,4-dioxan-10-one.

25: The method of claim 8, the therapeutically effective amount of the ampakine being about 10 mg/kg per day to about 50 mg/kg per day.

26: A method of treating non-neurodegenerative respiratory dysfunction in a subject associated with Rett syndrome, comprising:
chronically administering to the subject an amount of at least one ampakine effective to increase brain-derived neurotrophic factor expression in nodose sensory neurons of the subject and improve respiratory function of the subject, and
measuring respiratory activity in the subject following administration of the at least one ampakine.

27: The method of claim 26, wherein the ampakine is administered at an amount effective to enhance brain-derived neurotrophic factor signaling in the brain stem of the subject.

28: The method of claim 26, the ampakine being an allosteric modulator of the AMPA-receptor.

29: The method of claim 26, the ampakine comprising a compound having the formula: 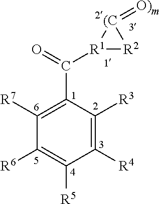
wherein,
R¹ is a member selected from the group consisting of N and CH;
m is 0 or 1;
R² is a member selected from the group consisting of (CR⁸₂)ₙ₋ₘ and Cₙ₋ₘR⁸₂₍ₙ₋ₘ₎₋₂, in which n is 4, 5, 6, or 7, the R⁸'s in any single compound being the same or different, each R⁸ being a member selected from the group consisting of H and C₁-C₆ alkyl, or one R⁸ being combined with either R³ or R⁷ to form a single bond linking the no. 3′ ring vertex to either the no. 2 or the no. 6 ring vertices or a single divalent linking moiety linking the no. 3′ ring vertex to either the no. 2 or the no. 6 ring vertices, the linking moiety being a member selected from the group consisting of CH₂, CH₂CH₂, CH═CH, O, NH, N(C₁-C₆ alkyl), N═CH, N═C(C₁-C₆ alkyl), C(O), O—C(O), C(O)—O, CH(OH), NH—C(O), and N(C₁-C₆ alkyl)-C(O);
R³, when not combined with any R⁸, is a member selected from the group consisting of H, C₁-C₆ alkyl, and C₁-C₆ alkoxy;
R⁴ is either combined with R⁵ or is a member selected from the group consisting of H, OH, and C₁-C₆ alkoxy;
R⁵ is either combined with R⁴ or is a member selected from the group consisting of H, OH, C₁-C₆ alkoxy, amino, mono(C₁-C₆ alkyl)amino, di(C₁-C₆ alkyl)amino, and CH₂OR⁹, in which R⁹ is a member selected from the group consisting of H, C₁-C₆ alkyl, an aromatic carbocyclic moiety, an aromatic heterocyclic moiety, an aromatic carbocyclic alkyl moiety, an aromatic heterocyclic alkyl moiety, and any such moiety substituted with one or more members selected from the group consisting of C₁-C₃ alkyl, C₁-C₃ alkoxy, hydroxy, halo, amino, alkylamino, dialkylamino, and methylenedioxy;
R⁶ is either H or CH₂OR⁹;
R⁴ and R⁵ when combined form a member selected from the group consisting of 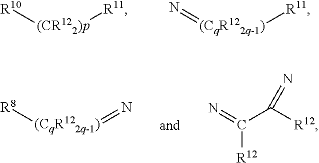
in which: R¹⁰ is a member selected from the group consisting of O, NH and N(C₁-C₆ alkyl);
R¹¹ is a member selected from the group consisting of O, NH and N(C₁-C₆ alkyl);
R¹² is a member selected from the group consisting of H and C₁-C₆ alkyl, and when two or more R¹²'S are present in a single compound, such R¹²'S are the same or different;
p is 1, 2, or 3; and
q is 1 or 2; and
R⁷, when not combined with any R⁸, is a member selected from the group consisting of H, C₁-C₆ alkyl, and C₁-C₆ alkoxy.

30: The method of claim 26, the ampakine comprising at least one of 1-(1,4-benzodioxan-6-ylcarbonyl)piperidine, 1-(quinoxalin-6-ylcarbonyl)piperidine, 2H,3H,6aH-pyrrolidino[2″,1″-3′,2″]1,3-oxazino[6′,5′-5,4]benzo[e]1,4-dioxan-10-one.

31: The method of claim 26, the therapeutically effective amount of the ampakine being about 10 mg/kg per day to about 50 mg/kg per day.
